(19) United States
(12) Patent Application Publication
(10) Pub. No.: US 2002/0197303 A1
(43) Pub. Date: Dec. 26, 2002

(54) Crude drug patch

(76) Inventor: Ki Hong Kim, Gangwon-do (KR)

Correspondence Address: JACOBSON HOLMAN PLLC, 400 SEVENTH STREET N.W., SUITE 600, WASHINGTON, DC, 20004, US

(21) Appl. No.: 10/151,901

(22) Filed: May 22, 2002

(30) Foreign Application Priority Data
Jun. 12, 2001 (KR) 2001-32958

(51) Int. Cl. A61K 35/78; A61K 9/70

(52) U.S. Cl. 424/449; 424/729; 424/757; 424/777

(57) ABSTRACT

The present invention relates to a crude drug patch, and in particular to a crude drug patch which is fabricated using a gardenia having a certain function such as an antimicrobial function, a suppression and fever removing function. A homeostasis function, etc., a raw rehmannia glutinose is capable of softening a coagulated blood, mitigating a blood stasis, and curing a wound. In addition, it is possible to mitigate a pain by sticking on a diseased part such as a labor pain/anti-inflammation, sprain, bruise, muscle pain, joint pain, lumbago, shoulder pain, neuralgia, rheumatic pain, etc. based on a combined prescription of a fermented soybean paste (toenjang), wheat flour and green tea at a certain ratio. Even when a crude drug patch according to the present invention is stuck on a skin using a natural material as an effective component, there is not any side effect.

### BACKGROUND OF THE INVENTION

1. Field of the Invention

The present invention relates to a crude drug patch, and in particular to a crude drug patch which is fabricated using a gardenia having a certain function such as an antimicrobial function, a suppression and fever removing function, a homeostasis function, etc., a raw rehmannia glutinose capable of softening a coagulated blood, mitigating a blood stasis, and curing a wound. In addition, it is possible to mitigate a pain by sticking a crude drug patch on a diseased part such as a labor pain/inflammation, sprain, bruise, muscle pain, joint pain, lumbago, shoulder pain, neuralgia, rheumatic pain, etc. based on a combined prescription of a fermented soybean paste (toenjang), wheat flour and green tea at a certain ratio. Even when a crude drug patch according to the present invention is stuck on a skin using a natural material as an effective component, there is no any side effect.

2. Description of the Background Art

In the conventional art, in order to medically treat a labor pain/anti-inflammation, sprain, bruise, muscle pain, joint pain, lumbago, shoulder pain, neuralgia, rheumatic pain, etc., an anodyne or a patch which is an internal medicine is generally used.

However, since the above internal medicine is an oral inoculation type medicine, a gastroenteric disorder may occur.

The above patch agent is a transdermal delivery type medicine, the agent is delivered for a relatively long time by a small amount. However, in the case of the patch agent which is widely used in the conventional art, the main components of the patch agent is an organic compound which is artificially fabricated in order for the main component of the agent to have a certain effect. In addition, in order to fabricate the above organic compound, the fabrication process is complicated, and it is expensive.

In order to overcome the above problems, the patch fabricated using the gardenia is disclosed in the Korean Utility model No. 20-0161528. However, since the above patch is fabricated using only the gardenia, the medical effects of the same are low.

In addition, a transdermal delivery agent fabricated using an extract of the gardenia is disclosed on the Korean Patent No. 96-0004017. However, since the above agent is fabricated mainly using the gardenia as an effective component, the medical effects of the same are low.

### SUMMARY OF THE INVENTION

Accordingly, it is an object of the present invention to provide a crude drug patch which overcomes the problems encountered in the conventional art.

It is another object of the present invention to provide a crude drug patch which is capable of fabricating a new crude drug patch agent based on a mixture of a gardenia and a raw rehmannia glutinose which are an oriental medical agent having a certain medical effect.

It is still another object of the present invention to provide a crude drug patch which is capable of preventing a side effect due to the use of an organic compound and directly sticking a patch on a diseased part by providing a patch agent which is easily obtained in our life, so that it is possible to efficiently mitigate a pain such as a labor pain/anti-inflammation, sprain, bruise, muscle pain, joint pain, lumbago, shoulder pain, neuralgia, rheumatic pain, etc.

In order to achieve the above objects, there is provided a crude drug patch which is formed of a raw rehmannia glutinose of 50˜200 weight % with respect to a gardenia of 100 weight %, a fermented soybean paste of 30˜120 weight % with respect to the gardenia of 100 weight %, and a wheat flour of 10˜120 weight % with respect to the gardenia of 100 weight %.

### BRIEF DESCRIPTION OF THE DRAWINGS

The present invention will become better understood with reference to the accompanying drawings which are given only by way of illustration and thus are not limitative of the present invention, wherein;

FIG. 1 is a schematic view illustrating the construction of a crude drug patch according to the present invention.

### DETAILED DESCRIPTION OF THE PREFERRED EMBODIMENTS

In a patch agent formed of a certain component including a gardenia, a crude drug patch according to the present invention is formed of a raw rehmannia glutinose of 50˜200 weight % with respect to a gardenia of 100 weight %, a fermented soybean paste of 30˜120 weight % with respect thereto, and a wheat flour of 10˜120 weight % with respect thereto.

The above crude drug patch according to the present invention will be described in detail.

The gardenia has been widely used as an oriental medicine material for curing a diuresis, eye disease, jaundice, etc. and is used as an effective component in the oriental medicine. The above gardenia is good for a gall, liver disease prevention, antimicrobial effect, anti-fungal effect, suppression and fever removing effect, hemostasis, etc. The above gardenia is extracted by a cool precipitation or heating method using a water solution or alcohol water solution and then is obtained using a lyophilization or decompression dried powder obtained by removing an insoluble extract or a flowing extract.

The thusly-obtained gardenia having the above-described medical effects is preferably included in a crude drug patch agent by 100 weight %. If the amount of the gardenia exceeds the above 100 weight %, the medical effect is decreased.

The raw rehmannia glutinose used as an effective component in the present invention is good for a heart, liver, etc. and has a strong replenish m and blood refreshing function and is effective for a body fluid loss and thirst. In addition, the raw rehmannia glutinose is capable of removing a subtropical state of the heart, helping a generation of blood, cooling the temperature of blood, lowering heats, softening hardened blood and mitigating the blood stasis. In addition, since the raw rehmannia glutinose is classified as a cooling component, the raw rehmannia glutinose is capable of implementing a hemostasis when a person's nose bleeds or people vomits blood from the mouth. In addition, in the case that there is a spot in the body, the raw rehmannia glutinose may be used for removing the spot in the body.

In the present invention, the above raw rehmannia glutinose is preferably included by 50˜200 weight % with respect to the gardenia of 100 weight %. If the amount of the raw rehmannia glutinose is below 50 weight %, it is impossible to obtain a desired medical effect. If the amount of the raw rehmannia glutinose is above 200 weight %, the stability is decreased when it is stored for a long time.

The fermented soybean paste used in the present invention is good for an anti-cancer effect, thrombus decomposition effect, antibody generation effect, detoxification operation, digestive operation, hypertension prevention effect, liver function enhancing effect, etc. In the present invention, the fermented soybean paste is used for a disinfection and neutralization of toxic. The fermented soybean paste is preferably included by 30˜120 weight % with respect to the gardenia of 100 weight %. If the amount of the fermented soybean paste is below 30 weight %, the medical effect is decreased, and if the amount of the fermented soybean paste is above 120 weight %, the stability may be decreased when storing the same for a long time.

In the present invention, the wheat flour is used as an adhesive base agent for fabricating the crude drug patch. The above wheat flour may be used for helping a penetration of agent effect by smoothing the skin when the patch is stuck on the skin and curing a small scar. In addition, it is possible to easily stick the patch on a skin or mucous membrane at a room temperature for thereby implementing an asepsis, disinfection, convergence and anesthesia, releasing and preventing a pain, smoothing a crust of a scar, effectively sucking a secretion and generating a new skin. The above wheat flour is preferably included by the amount of 10˜120 weight % with respect to the gardenia of 100 weight %. If the amount of the wheat flour is out of the above range, it is impossible to fabricate a desired patch. In addition, in the present invention, the starch may be used as a base agent.

The green tea used in the present invention is capable of implementing an anti-cancer operation, arteries hardening prevention operation, blood pressure increasing prevention operation, decayed teeth prevention operation, blood platelet coagulation prevention operation, anti-oxidation operation, aging prevention operation, heavy metal removing operation, etc. In the present invention, the green tea is basically directed to implementing a smell removing operation, aging prevention, antimicrobial operation with respect to various bacteria and an anti-inflammation operation. The green tea may be additionally added to the crude drug agent by 20˜30 weight % with respect to the gardenia of 100 weight %. If the amount of the green tea is below 20 weight %, it is impossible to implement a full smell removing operation, antimicrobial operation and anti-inflammation operation. If the amount of the green tea exceeds 30 weight %, the above effects are excellent, but it is impossible to obtain a desired stability when it is stored for a long time. In the present invention, as the effective components of the green tea, extracting using water or a proper organic solvent may use an extract or dried powder.

The crude drug patch agent according to the present invention is fabricated using the above materials for thereby obtaining a synergy effect based on an inter-cooperation between the materials. Therefore, in the present invention, it is possible to obtain the combined effects. The side effects are decreased, and the absorbing time of the effective components is extended for thereby implementing a long time medical effect.

The crude drug patch according to the present invention is fabricated by mixing a gardenia, raw rehmannia, fermented soybean paste, and green tea, adding a wheat flour to the mixture as an adhesive base material, coating the above mixture on a surface of a mold having a certain size by a thin thickness for thereby fabricating the crude drug patch.

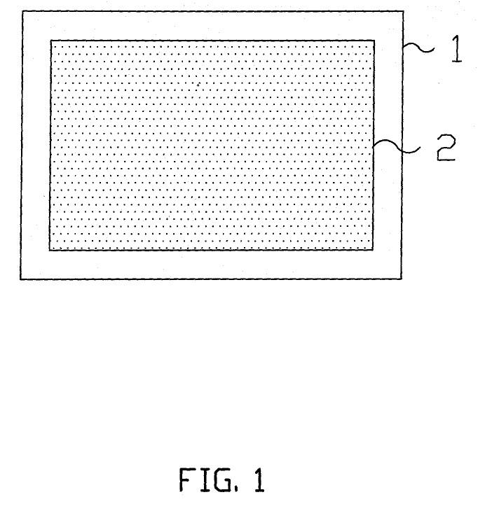

As shown in FIG. 1, the crude drug patch according to the present invention is formed of a curing agent layer 1, and a hetero-member 2. The crude drug patch agent according to the present invention has a certain viscosity, so that it is possible to easily stick on a diseased part of a body. In order to prevent the diseased part from being exposed to the outside and implement a long time continuous effect of the agent, the patch is stuck on the diseased part, and then the stuck patch is stably fixed using a certain sticking member.

In addition, the crude drug patch agent according to the present invention may be fabricated by adding an absorption agent, viscosity increasing agent and additive which are conventionally used for fabricating a conventional patch agent.

In addition, the crude drug patch agent according to the present invention is easily fabricated using a gardenia and raw rehmannia which are easily obtained in our life. In addition, since the above gardenia and raw rehmannia are edible, even when the crude drug patch fabricated using the gardenia and raw rehmannia is stuck on the skin of a human body, there is not any side effect. In addition, the crude drug patch according to the present invention is very effective for mitigating the pain of a headache, toothache and joint muscle pain. Furthermore, the crude drug patch according to the present invention is very medically effective for curing a skin inflammation based on an anti-inflammation effect, suppression and anti-microbial operation.

The preferred examples of the present invention will be described in detail. The following descriptions are not limited to it.

### EXAMPLE

As shown in Table 1, a gardenia, raw rehmannia glutinose, and fermented soybean paste are mixed, and wheat flour is added to the mixture of the same. A resultant mixture is coated on a surface of a mold having a certain size and is dried for thereby fabricating a crude drug patch according to the present invention.

TABLE 1

| | Component (g) | | | | |
|---|---|---|---|---|---|
| | | Raw | Fermented | | |
| | | rehmannia | soybean | Wheat | Green |
| | Gardenia | glutinose | paste | flour | tea |
| Example 1 | 20 | 20 | 20 | 20 | — |
| Example 2 | 20 | 40 | 10 | 10 | — |
| Example 3 | 20 | 10 | 20 | 24 | 4 |
| Example 4 | 20 | 20 | 15 | 15 | 6 |

The crude drug patch according to the present invention and the Kefen(a product of Jeil pharmacy company(Korea) Which is commercially available in life and is a comparison product of the crude drug patch according to the present invention were stuck on the skins of the patients who were suffering from a bruise and muscle pain. The results were checked after 1^{st}, 3^{rd}, and 5^{th} days were passed. Tables 2(bruise) and Table 3(muscle pain) show a result of the above comparison.

As seen in Table 2 and Table 3, the marks “+++” represent the cured state of the bruise and muscle pain, and the marks “−−−” represent the suffering state due to the bruise and muscle pain.

TABLE 2

| | A | | | B | | | C | | | D | | |
|---|---|---|---|---|---|---|---|---|---|---|---|---|
| | 1^{st} | 3^{rd} | 5^{th} | 1^{st} | 3^{rd} | 5^{th} | 1^{st} | 3^{rd} | 5^{th} | 1^{st} | 3^{rd} | 5^{th} |
| | day | day | day | day | day | day | day | day | day | day | day | day |
| Example 1 | ++ | +++ | +++ | +++ | +++ | +++ | ++ | +++ | +++ | +++ | +++ | +++ |
| Example 2 | +++ | +++ | +++ | ++ | +++ | +++ | +++ | +++ | +++ | ++ | +++ | +++ |
| Example 3 | +++ | +++ | +++ | +++ | +++ | +++ | ++ | +++ | +++ | +++ | +++ | +++ |
| Example 4 | ++ | +++ | +++ | ++ | ++ | +++ | +++ | +++ | +++ | +++ | +++ | +++ |
| Compared | + | ++ | +++ | + | + | ++ | + | ++ | ++ | + | ++ | ++ |
| product | | | | | | | | | | | | |
| (Kefen) | | | | | | | | | | | | |
| Patch not | — | — | — | — | — | — | — | — | — | — | — | — |
| stuck | | | | | | | | | | | | |

TABLE 3

| | A | | | B | | | C | | | D | | |
|---|---|---|---|---|---|---|---|---|---|---|---|---|
| | 1^{st} | 3^{rd} | 5^{th} | 1^{st} | 3^{rd} | 5^{th} | 1^{st} | 3^{rd} | 5^{th} | 1^{st} | 3^{rd} | 5^{th} |
| | day | day | day | day | day | day | day | day | day | day | day | day |
| Example 1 | ++ | +++ | +++ | +++ | ++ | +++ | ++ | +++ | +++ | +++ | +++ | +++ |
| Example 2 | +++ | +++ | +++ | ++ | +++ | +++ | +++ | +++ | +++ | +++ | +++ | +++ |
| Example 3 | ++ | +++ | +++ | +++ | +++ | +++ | +++ | +++ | +++ | +++ | +++ | +++ |
| Example 4 | +++ | +++ | +++ | ++ | +++ | +++ | +++ | +++ | +++ | ++ | +++ | +++ |
| Compared | ++ | ++ | +++ | ++ | ++ | ++ | ++ | ++ | +++ | + | ++ | +++ |
| product | | | | | | | | | | | | |
| (Kefen) | | | | | | | | | | | | |
| Patch not | — | — | — | — | — | — | — | — | — | — | — | — |
| stuck | | | | | | | | | | | | |

As shown in the above tables 2 and 3, the crude drug patch according to the first through fourth examples of the present invention has an excellent medical effect by sticking the patch on a diseased part such as a muscle pain and bruise compared to the compared product.

As described above, the crude drug patch according to the present invention is fabricated using the crude drug agents which are easily obtained in life. The crude drug agents are edible, so that the patch made of the crude drug agents is stuck on a skin, there is not any side effect. Therefore, it is possible to quickly mitigate the pains by sticking the crude drug patch on a diseased part such as a labor pain/anti-inflammation, sprain, bruise, muscle pain, joint pain, lumbago, shoulder pain, neuralgia, rheumatic pain, etc.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

What is claimed is:

1. In a crude drug patch which is formed of a certain component including a gardenia, a crude drug patch which is formed of a raw rehmannia glutinose of 50˜200 weight % with respect to a gardenia of 100 weight %, a fermented soybean paste of 30˜120 weight % with respect to the gardenia of 100 weight %, and a wheat flour of 10˜120 weight % with respect to the gardenia of 100 weight %.

2. The patch of claim 1, wherein said crude drug patch further includes a green tea of 20˜30 weight % with respect to a gardenia of 100 weight %.
